(19) United States
(12) Patent Application Publication
(10) Pub. No.: US 2026/0088173 A1
(43) Pub. Date: Mar. 26, 2026

(54) EVALUATING FAITHFULNESS OF EXPLAINABLE AI FOR MEDICAL DECISION MAKING

(71) Applicant: NEC Laboratories America, Inc., Princeton, NJ (US)

(72) Inventors: Wei Cheng, Princeton Junction, NJ (US); Xu Zheng, Miami, FL (US); Haifeng Chen, West Windsor, NJ (US); Dongsheng Luo, Miami, FL (US)

(21) Appl. No.: 19/330,013

(22) Filed: Sep. 16, 2025

Related U.S. Application Data
(60) Provisional application No. 63/699,337, filed on Sep. 26, 2024.

(51) Int. Cl. G16H 50/20; G16H 10/60

(52) U.S. Cl. CPC G16H 50/20; G16H 10/60

(57) ABSTRACT

Methods and systems include fine-tuning a classifier while masking part of a training dataset to cause a distribution of the classifier to match a distribution of an explainer model. A performance of the explainer model is determined using the fine-tuned classifier to ensure that the explainer has an above-threshold fidelity. A downstream task is performed using the classifier and the explainer model.

### RELATED APPLICATION INFORMATION

This application claims priority to U.S. Patent Application No. 63/699,337, filed on Sep. 26, 2024, incorporated herein by reference in its entirety.

### BACKGROUND

### Technical Field

The present invention relates to machine learning and, more particularly, to determining the faithfulness of AI explanations.

### Description of the Related Art

Explainable artificial intelligence (AI) models generate an output, such as a classification, and furthermore identify information that serves to explain how they reached their conclusion. One type of AI explanation uses post-hoc instance-level explanation, where, given a pre-trained classifier with a specific input, the explanation identifies the most important features of the model's output. For example, such an explanation may identify a set of important pixels in an image that led to a particular classification.

### SUMMARY

A method includes fine-tuning a classifier while masking part of a training dataset to cause a distribution of the classifier to match a distribution of an explainer model. A performance of the explainer model is determined using the fine-tuned classifier to ensure that the explainer has an above-threshold fidelity. A downstream task is performed using the classifier and the explainer model.

A system includes a hardware processor and a memory that stores a computer program. When executed by the hardware processor, the computer program causes the hardware processor to fine-tune a classifier while masking part of a training dataset to cause a distribution of the classifier to match a distribution of an explainer model. A performance of the explainer model is determined using the fine-tuned classifier to ensure that the explainer has an above-threshold fidelity. A downstream task is performed using the classifier and the explainer model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of an explainer model fidelity determination for a classifier, in accordance with an embodiment of the present invention;

FIG. 2 is pseudo-code for determining a fidelity score for an explainer model, in accordance with an embodiment of the present invention;

FIG. 3 is a block/flow diagram for evaluating, selecting, and using an explainer model for a downstream task, in accordance with an embodiment of the present invention;

FIG. 4 is a block diagram of a healthcare facility that uses a classifier with high-fidelity AI explanation, in accordance with an embodiment of the present invention;

FIG. 5 is a block diagram of a computing device that selects an explainer model for a classifier, in accordance with an embodiment of the present invention;

FIG. 6 is a diagram of a neural network architecture that can be used as part of a classifier model, in accordance with an embodiment of the present invention; and

FIG. 7 is a diagram of a deep neural network architecture that can be used as part of a classifier model, in accordance with an embodiment of the present invention.

### DETAILED DESCRIPTION OF PREFERRED EMBODIMENTS

The faithfulness of an explanation for an artificial intelligence (AI) model can be determined using fine-tuned surrogate models. A consistent evaluation strategy enhances the robustness of the faithfulness measurement and provides a clear understanding of the relationship between the explanation size and the evaluation accuracy.

A fine-tuned fidelity framework addresses out-of-distribution challenges when evaluating the faithfulness of an explanation. The surrogate models use the same augmentation process in fine-tuning and evaluation stages, ensuring that the evaluation inputs remain within the same distribution. This enhances the reliability of the faithfulness assessments and provides a consistent evaluation strategy across varying levels of explanation sparsity.

The fine-tuning strategy is explanation-agnostic to prevent information leakage. A controlled random masking operation is further used to overcome out-of-distribution problems. The fine-tuning process uses stochastic masking operations, such as randomly dropping pixels in images, tokens in language, or time steps in time series data, to generate augmented training samples. The augmented training data is then used to fine-tune the surrogate model. During an evaluation phase, a removal strategy generates stochastic masks conditioned on an explainer's output, designed to be in-distribution with respect to the masks used in the fine-tuning.

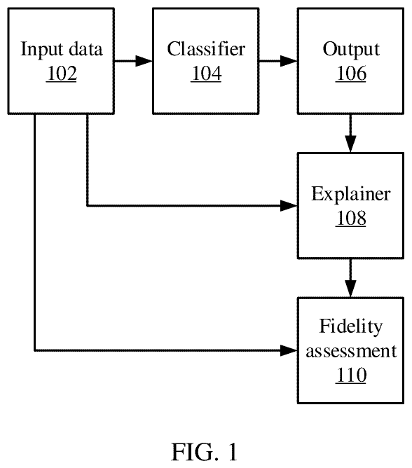

Referring now to FIG. 1, an explainable AI system is shown. Input data 102 is processed by a classifier 104 to generate an output 106. It should be understood that the classifier 104 is used solely for the sake of explanation and that any appropriate machine learning task may be performed instead. For example, an image input may be classified to determine what object is shown within the image, or to determine whether a face is present. In another example, the input data 102 may be time series information relating to a patient's health condition, and the classifier 104 may output a diagnosis. Output 106 may thus include information about the contents of the input data 102.

An explainer 108 is similarly implemented as a machine learning model which uses the input data 102 and information from the classifier 104 to identify which parts of the input data 102 most strongly affected the output 106. Following the example of image classification, the explainer 108 may identify pixels in the input data 102 that include a detected object. Following the example of diagnosing a patient's health condition, the explainer 108 may identify periods of the time in the time series data which indicate the health condition, or identify specific time series within a multivariate time series that point to the diagnosis. In some embodiments, the explainer 108 may be part of the same machine learning model as the classifier 104. Fidelity assessment 110 may be used to determine how accurate the explainer's explanations are.

A classification model f:X→Y, such as a neural network, takes an input X∈ [symbol] ^{t×d} and outputs a label Y∈ [symbol] , where [symbol] is a finite set of labels, t=h×w is a number of pixels for an input image having height h and width w, and d is a number of channels per input pixel. Analogously, in natural language processing and time-series classification tasks, t∈ [symbol] represents the time index, and d is the feature dimension.

An explanation function (explainer) consists of a pair of mappings ψ=(φ, ξ), where φ:

$$X ↦ {ℝ}_{+}^{t \times d}$$

is the score function, mapping each input element to its (nonnegative) importance score, and a mask function ξ: φ(X) [symbol] M, mapping the output of the score function to a binary mask M∈{0, 1}^{t×d}. The masked input X⊙M is called the explanation for the input X and model f(⋅), where ⊙ represents elementwise multiplication. The explanation size is S=∥M∥₁, where ∥⋅∥₁ is the [symbol] ₁ norm. That is, the explanation size S is the number of non-zero elements of M. In general, the size may be deterministically set to a constant value s, or alternatively, it may depend on the output of the score function, e.g., input elements receiving a score higher than a given threshold are included in the mask and the rest are removed.

Assuming that the (ground-truth) data distribution is P_{X,Y}, then a good explainer is one which minimizes the total variation distance d_{TV}(P_{Y|X}, P_{Y|X⊙M}), while satisfying an explanation size constraint [symbol] _{X}(∥M∥₁)≤s, where s∈ [symbol] is the desired average explanation size. The minimization of the total variation essentially enforces that the posterior distribution of the classifier output be mostly determined by the masked input explanation, implying that the subset of input components which are removed by the mask have a low influence on the classifier output.

The performance of an explainer can be formally quantified in terms the total variation distance d_{TV}(P_{Y|X}, P_{Y|X⊙M}) as a function of the average explanation size [symbol] _{X}(∥M∥₁). However, in most problems of interest, the underlying statistics P_{X,Y} are not available, and hence direct evaluation of the total variation distance is not possible. Some datasets are accompanied by ground-truth explanations, which enables the use of measures for evaluating the explainers' quality. However, the ground-truth explanations are available only for a limited collection of datasets, and even when ground-truth explanations are available, they may not accurately reflect the model's internal decision-making processes.

A fidelity metric based in graph domain removal may be given the input and label pair (x, y) and a binary mask m, with the metrics being defined as follows:

$${Fid}^{+} ( ψ ) = \frac{1}{\vert 𝒯 \vert} {\sum }_{( {x}_{i} , {y}_{i} ) ∈ 𝒯} 𝕝 ( {y}_{i} = f ( {x}_{i} ) ) - 𝕝 ( {y}_{i} = f ( {x}_{i} - {x}_{i} ⊙ {m}_{i} ) )$$

$$Fi {d}^{-} ( ψ ) = \frac{1}{\vert 𝒯 \vert} {\sum }_{( {x}_{i ,} {y}_{i} ) ∈ 𝒯} 𝕝 ( {y}_{i} = f ( {x}_{i} ) ) - 𝕝 ( {y}_{i} = f ( {x}_{i} ⊙ {m}_{i} ) )$$

where [symbol] is the dataset used for evaluating the performance of the explainer, n is the size of the dataset, [symbol] (⋅) denotes the indicator function, and mᵢ=ψ(xᵢ) is the explanation corresponding to xᵢ produced by the explainer ψ(⋅). Here, Fid⁺ measures prediction changes when removing important features, while Fid⁻ evaluates model performance when keeping only important features.

When elements are removed from an input, whether they are pixels in images, time steps in time series, or edges in graphs, the modified input may no longer follow the original data distribution that the model was trained on. For example, when evaluating image explanations by zeroing out important pixels, the resulting images with black patches are unlikely to resemble natural images. Consequently, the model's predictions on these modified inputs may be unreliable, not due to low quality of the explanation itself, but because the model is operating outside its training distribution.

The Fidelity metric highly relies on the robustness of the underlying classifier to removal of potentially large sections of the input, e.g., the removal of a large subgraph explanation for Fid⁺ or its complement for Fid⁻. That is, the classifier should be robust to out-of-distribution inputs for the Fidelity metric to align with those of the (theoretically justified) total-variation-based metric. Rather than retraining the model, R-Fidelity introduces a stochastic removal strategy that addresses the out-of-distribution issue by controlling the size of removed sections and randomly sampling which elements to remove, thus limiting the distribution shift of perturbed inputs. Specifically, the following Robust Fidelity metrics (RFid) may be expressed as:

$${RFid}^{+} ( ψ , {α}^{+} , s ) = \frac{1}{\vert 𝒯 \vert} {\sum }_{( {x}_{i} , {y}_{i} ) ∈ 𝒯} 𝕝 ( {y}_{i} = f ( {x}_{i} ) ) - P ( {y}_{i} = f ( {χ}^{+} ( {x}_{i} , {α}^{+} , s )$$

$${RFid}^{-} ( ψ , {α}^{-} , s ) = \frac{1}{\vert 𝒯 \vert} {\sum }_{( {x}_{i} , {y}_{i} ) ∈ 𝒯} 𝕝 ( {y}_{i} = f ( {x}_{i} ) ) - P ( {y}_{i} = f ( {χ}^{-} ( {x}_{i} , {α}^{-} , s ) ) ,$$

where χ⁺(xᵢ, α⁺, s) is a sampling function which randomly, uniformly, and independently removes └sα⁺┘ elements from the s highest scoring elements of xᵢ based on the scores produced by φ(xᵢ), and χ⁻(xᵢ, α⁻) removes ┌(td−s)α⁻┐ elements from the lowest scoring td−s elements. If α⁺=α⁻=1, then the RFid metric reduces to the Fid metric. On the other hand, as α⁺ and α⁻ are decreased, fewer input elements are removed, hence requiring lower out-of-distribution robustness to ensure the accuracy of the evaluation output.

A significant limitation of prior explanation evaluation metrics is the loss in accuracy due to the out-of-distribution nature of the modified inputs generated by the application of removal strategies. For instance, the probability difference P(Y=f(X))−P(Y=f(X−X⊙(M)) may be large, even for low-quality explanations. This occurs because the modified input X−X⊙M is OOD for the trained classifier f(⋅), despite P_{Y|X} and P_{Y|X-X⊙M} being close to each other. Consequently, this yields a high Fid⁺ score despite the explanation's low quality with respect to the theoretically justified total variation metric.

A partial solution in the graph domain addresses this issue by removing only an α⁺ fraction of the explanation subgraph and α⁻ fraction of the non-explanation subgraphs. However, two issues degrade the evaluation quality of the RFid metric. First, the classifier may lack robustness and produce unreliable outputs even when the input is only slightly perturbed. Second, if the original explanation size is large (small), then removing an α⁺or α⁻ of the explanation (non-explanation) part of the input, this would still yield out-of-distribution inputs.

To that end, F-Fidelity may be used as a metric for robust evaluation of explainable AI systems. The model is fine-tuned with randomly masked inputs to improve its robustness to perturbation. Then a controlled stochastic removal process ensures the perturbed inputs remain within the distribution seen during fine-tuning.

To achieve reliable predictions on partially removed inputs, we design a fine-tuning process that randomly removes up to β∈[0, 1] ratio of input elements. To elaborate, we introduce a stochastic mask generator P_{β}:(t, d) [symbol] M_{β}, which takes the input dimensions (t, d) as input and outputs a mask M_{β}∈{0, 1}^{t×d} of size βtd, with up to βtd non-zero elements. For example, in image classification, the mask generator is designed to select random image pixels or patches for removal. Formally, the fine-tuning loss as may be expressed as:

$$L = {𝔼}_{X , Y} [ ℒ ( f ( X - {P}_{β} ⊙ X ) , Y ) ] ,$$

where [symbol] is a loss function used during training, such as a cross-entropy loss.

The RFid metric is modified to ensure consistency with the fine-tuning strategy by upper-bounding the total number of removed elements by βtd, the same bound used during fine-tuning. That is, for a fixed β, and RFid parameters

$${α}_{orig}^{+} , {α}_{orig}^{-} ∈ [ 0 , 1 ] ,$$

the upper-bounded RFid parameters may be set as:

$${α}^{+} = min ( {α}_{orig}^{+} , \frac{β t d}{s} ) and {α}^{-} = min ( {α}_{orig}^{-} , \frac{β t d}{( t d - s )} ) ,$$

so that the sampling functions χ⁺ and χ⁻ remove the minimum of

$${α}_{orig}^{+} s$$

(based on explanation size) and βtd (based on input size) elements for χ⁺, and the minimum of

$${α}_{orig}^{-} ( td - s )$$

and βtd elements for χ⁻, providing absolute upper bounds on the number of removed elements.

The resulting metrics, which use the fine-tuning process and the RFid⁺ and RFid⁻ metrics with sampling rates that are truncated based on β, as

$${FFid}^{+} ( ψ , {α}_{orig}^{+} , β , s ) and$$

$$FFi {d}^{-} ( ψ , {α}_{orig}^{-} , β , s ) ,$$

respectively. Both FFid⁺ and FFid⁻ can take negative values in certain cases. This occurs when the accuracy after masking exceeds the original prediction accuracy. Alternative formulations could enforce positive values by only reporting masked accuracy and deletion/insertion scores.

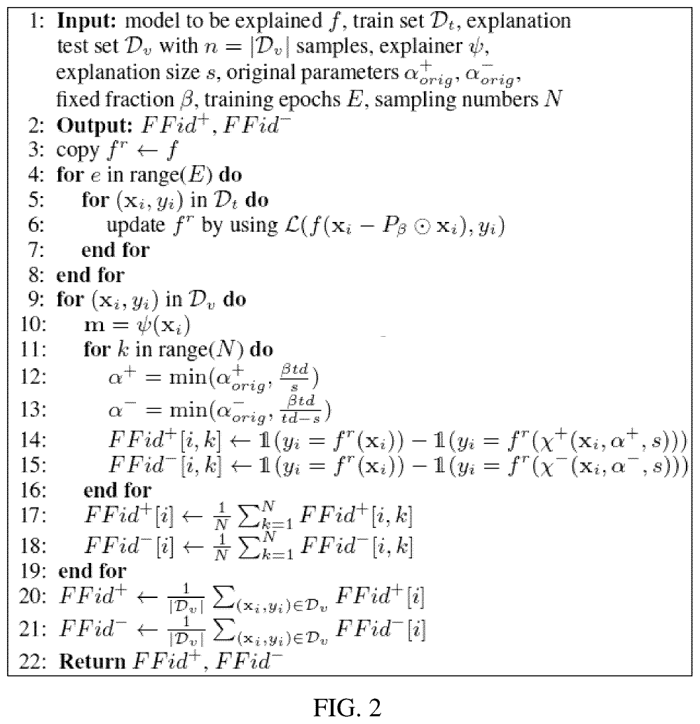

Referring now to FIG. 2, pseudo-code is provided that computes the metrics FFid⁺ and FFID⁻. The model f and explainer ψ are used as input and the process loops over the different data elements in the training dataset [symbol] ₜ, updating values of the model using the training loss function [symbol] . Then, for elements in the validation dataset [symbol] ᵥ, explanations m are generated.

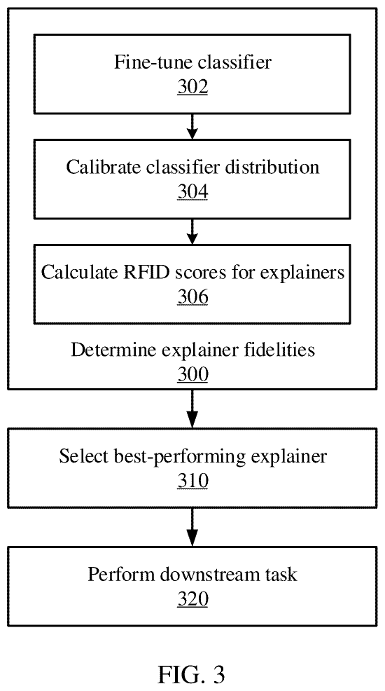

Referring now to FIG. 3, a method of determining and using the fidelity of an explanation is shown. Block 300 determines explainer fidelity performance for a set of different explainer models. Block 310 selects the best-performing explainer (e.g., the explainer having the highest fidelity) and block 320 then performs a downstream task using the selected explainer.

Determining the explainer fidelities fine-tunes 302 a classifier model 104, using a training loss function with labeled training data. Block 304 calibrates the fine-tuned classifier to prevent out-of-distribution problems and block 306 calculates RFid⁺ and RFid⁻ scores for each of the explainer models.

As shown, block 310 may select between multiple explainer models to identify a best-performing model. However, the present principles may also be applied to a single explainer model, for example comparing the FFid scores to a threshold value to determine whether the explainer model has a minimum level of fidelity before using it.

Block 320 uses the best-performing explainer model 108 to identify the functional features of input data in the downstream task that take the majority part in the decision making of the classifier 104. As noted above, this may identify pixels in an input image, time ranges and variables in multivariate time series data, or the functional subgraph structure of a chemical compound for drug discovery.

Block 320 may generate this explanation with an appropriate size or scope. Ground truth explanations would ideally be discretized into distinct clusters representing different levels of importance. For example, in image classification, pixels associated with the target object tend to receive high importance scores. Conversely, pixels corresponding to the background or irrelevant regions receive low scores. However, in many practical scenarios, even good explainers that produce accurate explanation masks, as measured by the Fid and RFid evaluation metrics, may yield explanation scores that are not discretized into distinct clusters.

The FFid metric can recover the cluster sizes given an explainer that outputs the correct explanation mask (i.e., correctly ranks the importance of input elements). Thus, provided the explainer outputs an accurate mask function, FFid can recover the explanation size (also known as sparsity).

A classification task may be defined by a joint distribution P_{X,Y} and a classifier f:x [symbol] y. The input elements can be partitioned into several influence tiers. That is, for any given input x, there exists a partition [symbol] ₖ(x), k∈[r] of the index set [t]×[d], where [symbol] ₖ(x) represents the set of indices of the input elements belonging to tier k, and cₖ=| [symbol] ₖ(x)| are the (fixed) tier sizes. For a given mask m, the probability of correct classification based on the masked input x⊙m depends only on the counts of unmasked elements in each influence tier. Formally, P(Y|x⊙m)=g(j₁, j₂, . . . , jᵣ), where g:[c₁]×[c₂]× . . . ×[cᵣ]→[0, 1] is a function monotonically increasing with respect to the lexicographic ordering on its input, and jₖ∈[cₖ] is the number of elements in [symbol] ₖ(x) whose corresponding mask element in m is non-zero (unmasked). Shapley-value-based explanations provide a theoretical foundation for this analysis. Given label y, the Shapley value associated with an element (i, j)∈[t]×[d] of x is given as:

$${S}_{x} ( i , j ) = {\sum }_{m : {m}_{i , j} = 0} \frac{{‖ m ‖}_{1} ! ( td - {‖ m ‖}_{1} - 1 ) !}{( td ) !} ( P ( Y = y \vert x ⊙ {m}^{′} ) - P ( Y = y \vert x ⊙ m ) ) ,$$

where m′ is the mask obtained from m by setting

$${m}_{i , j}^{′} = 1$$

(unmasking the (i, j) element). Under the aforementioned influence tier assumption, it is straightforward to verify that input elements within the same influence tier receive equal Shapley values. Specifically, for any k∈[r] and any (i, j), (i′, j′)∈ [symbol] ₖ(x), Sₓ(i, j)=Sₓ(i′, j′). For this classification task, and a given pre-trained classifier f(⋅), a Shapley-value-based explainer may be expressed as ψ(⋅). For

$${α}_{orig}^{+} ∈ [ 0 , 1 ]$$

and β∈[0, α⁺], let

$$e ( s ) = {𝔼}_{X , Y} ( {FFid}^{+} ( ψ , {α}_{orig}^{+} , β , s ) , s ∈ [ 0 , td ] .$$

Then, e(s) is monotonically increasing for s∈[0, c₁] and monotonically decreasing for

$$s ∈ [ max ( \frac{β}{{a}_{orig}^{+}} td , {c}_{1} ) , td ] .$$

This shows that FFid⁺ can recover the size of the most influential tier (i.e., the first cluster size) when the explainer's ranking is close to that of an ideal Shapley-based explainer. Specifically, the value of s in which FFid⁺ changes direction corresponds to the size of an influence tier. This result implies that even when an explainer provides continuous scores without distinct clustering, FFid can infer the underlying discrete structure of the ground truth explanations and recover the explanation size.

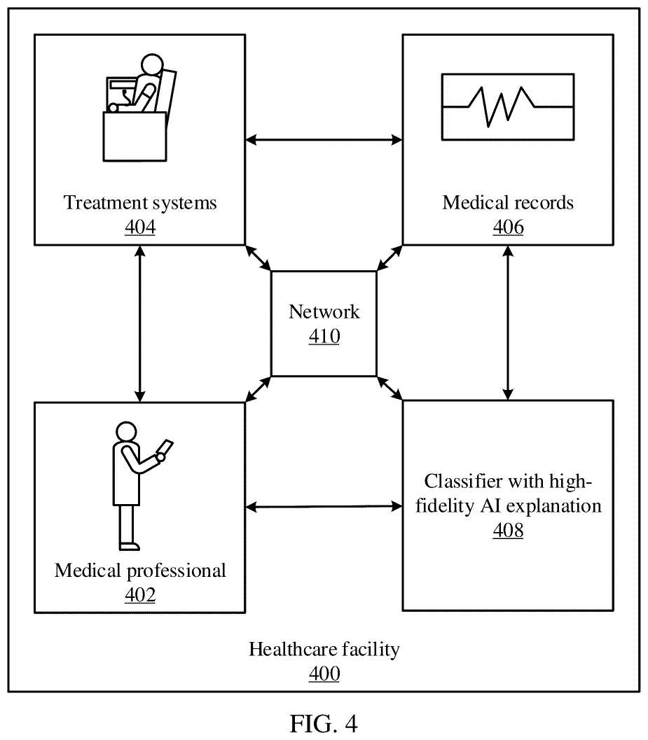

Referring now to FIG. 4, a diagram of time series analysis is shown in the context of a healthcare facility 400. A classifier with high-fidelity AI explanation 408 may be used to aid in medical decision making, for example by identifying a time or biometric that relates to a diagnosis. The classifier may take multivariate time series information from the medical records 406 of the patient and generate a diagnosis, with an explanation that indicates a particular period of time and type of sensor data that led to the diagnosis. This information can help to provide confidence in the diagnosis and may indicate avenues for further testing and treatment.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

The classifier with high-fidelity AI explanation 408 may be used to diagnose the patient's medical condition, for example classifying the input data to determine what is causing the patient's symptoms, indicated by the medical records 406 and input from medical professionals 402. The corresponding explanation can be relied on to provide a correct explanation of the diagnosis, without risking errors that might otherwise be caused by scenarios that are out of the distribution of the explainer's training data.

The different elements of the healthcare facility 400 may communicate with one another via a network 410, for example using any appropriate wired or wireless communications protocol and medium. Thus the classifier with high-fidelity AI explanation 408 receives data from treatment systems 404, medical professionals 402, and from medical records 406, and generates its diagnosis and explanation based on these diverse inputs. The classifier 408 may further coordinate with treatment systems 404 in some cases to automatically administer or alter a treatment. For example, the diagnosis of the classifier 408 may be used to determine a course of treatment, automatically administering therapeutic medications via the treatment systems 404.

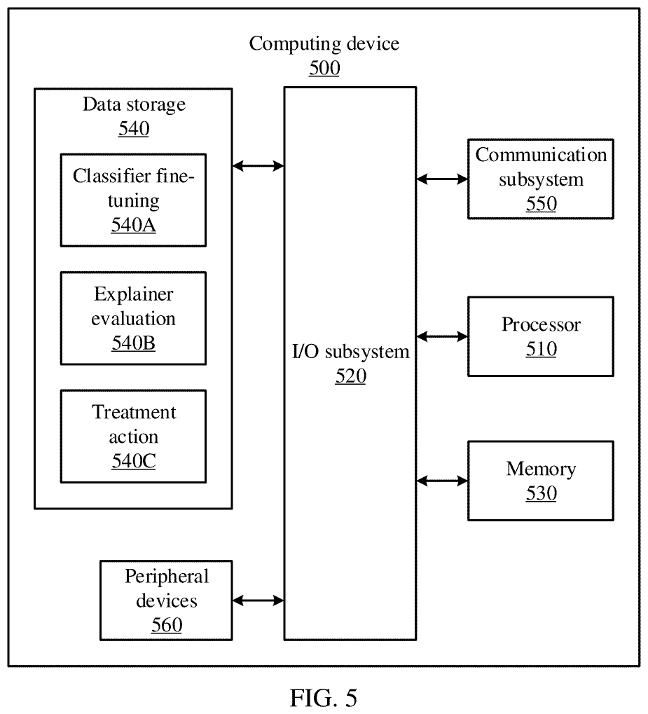

Referring now to FIG. 5, an exemplary computing device 500 is shown, in accordance with an embodiment of the present invention. The computing device 500 is configured to perform a machine learning classification with high-fidelity explanation.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for fine-tuning a classifier, 540B for evaluating explainer models, and/or 540C for performing treatment actions. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

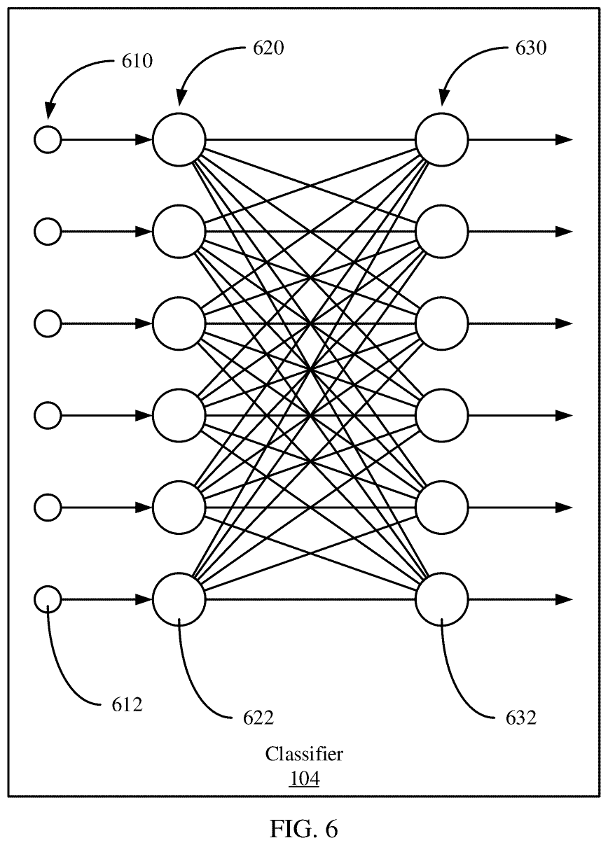

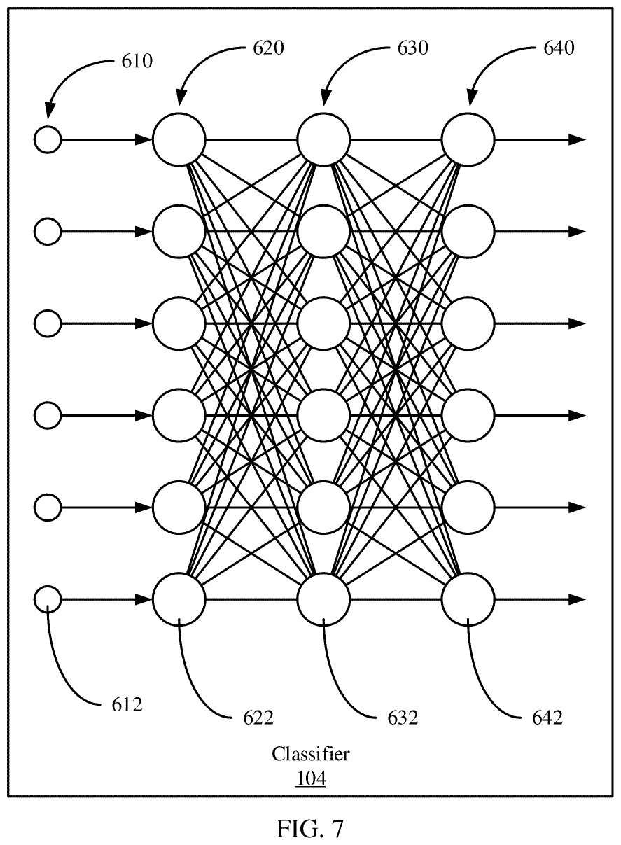

Referring now to FIGS. 6 and 7, exemplary neural network architectures are shown, which may be used to implement parts of the present machine learning models, such as the classifier 104. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
fine-tuning a classifier while masking part of a training dataset to cause a distribution of the classifier to match a distribution of an explainer model;
determining a performance of the explainer model using the fine-tuned classifier to ensure that the explainer has an above-threshold fidelity; and
performing a downstream task using the classifier and the explainer model.

2. The method of claim 1, wherein masking part of the training dataset includes masking a random portion of elements in training samples of the training dataset.

3. The method of claim 1, wherein the performance is determined as a robust fidelity metric with truncated sampling rates.

4. The method of claim 2, wherein the performance is enforced to have positive values by only reporting masked accuracy and deletion/insertion scores.

5. The method of claim 1, further comprising fine-tuning the classifier while masking part of a training dataset to cause a distribution of the classifier to match a distribution of one or more additional explainer models.

6. The method of claim 5, wherein determining the performance of the explainer model further determines the performance of the one or more additional explainer models, wherein the downstream task is performed using a selected model from the explainer model and the one or more additional explainer models having a highest performance.

7. The method of claim 1, wherein performing the downstream task is done using the classifier after fine-tuning.

8. The method of claim 1, wherein the downstream task includes medical information relating to a patient's health condition.

9. The method of claim 8, wherein the classifier accepts multivariate time series data of medical records of the patient as an input and performs a diagnosis based on the multivariate time series data, and wherein the explainer identifies a portion of the multivariate time series data that supports the diagnosis to assist in medical decision making.

10. The method of claim 9, further comprising automatically performing a treatment action on the patient responsive to the diagnosis.

11. A system, comprising:
a hardware processor; and
a memory that stores a computer program which, when executed by the hardware processor, causes the hardware processor to:
fine-tune a classifier while masking part of a training dataset to cause a distribution of the classifier to match a distribution of an explainer model;
determine a performance of the explainer model using the fine-tuned classifier to ensure that the explainer has an above-threshold fidelity; and
perform a downstream task using the classifier and the explainer model.

12. The system of claim 11, wherein the masking of part of the training dataset includes masking a random portion of elements in training samples of the training dataset.

13. The system of claim 11, wherein the performance is determined as a robust fidelity metric with truncated sampling rates.

14. The system of claim 12, wherein the performance is enforced to have positive values by only reporting masked accuracy and deletion/insertion scores.

15. The system of claim 11, wherein the computer program further causes the hardware processor to fine-tune the classifier while masking part of a training dataset to cause a distribution of the classifier to match a distribution of one or more additional explainer models.

16. The system of claim 15, wherein the determination of the performance of the explainer model further determines the performance of the one or more additional explainer models, wherein the downstream task is performed using a selected model from the explainer model and the one or more additional explainer models having a highest performance.

17. The system of claim 11, wherein performance of the downstream task is done using the classifier after fine-tuning.

18. The system of claim 11, wherein the downstream task includes medical information relating to a patient's health condition.

19. The system of claim 18, wherein the classifier accepts multivariate time series data of medical records of the patient as an input and performs a diagnosis based on the multivariate time series data, and wherein the explainer identifies a portion of the multivariate time series data that supports the diagnosis to assist in medical decision making.

20. The system of claim 19, wherein the computer program further causes the hardware processor to automatically perform a treatment action on the patient responsive to the diagnosis.
